(19) United States
(12) Patent Application Publication
(10) Pub. No.: US 2025/0152680 A1
(43) Pub. Date: May 15, 2025

(54) COMPOSITIONS COMPRISING GLUCOCEREBROCIDASE AND METHODS OF USE THEREOF

(71) Applicant: M6P THERAPEUTICS, INC., St. Louis, MO (US)

(72) Inventors: Robert Russell GOTSCHALL, Doylestown, PA (US); Lin LIU, Ballwin, MO (US)

(21) Appl. No.: 18/835,789

(22) PCT Filed: Feb. 7, 2023
(86) PCT No.: PCT/US2023/012523

§ 371 Date: Aug. 5, 2024

(51) Int. Cl. A61K 38/47; A61P 3/00; C12N 9/24

(52) U.S. Cl. CPC A61K 38/47; A61P 3/00; C12N 9/2402; C12Y 302/01045

(57) ABSTRACT

Provided are compositions comprising glucocerebrosidase having increased phosphorylation of High Mannose and Hybrid N-linked Oligosaccharides. Also provided herein are methods of treating Gaucher disease comprising administering to a subject the compositions of the disclosure.

### CROSS REFERENCE TO RELATED APPLICATIONS

This application is a 371 of International Application No. PCT/US2023/12523, filed Feb. 7, 2023, which claims the benefit of priority to U.S. Patent Application Ser. No. 63/307,464, filed Feb. 7, 2022, the entire contents of which are incorporated herein by reference.

### BACKGROUND

Lysosomes are organelles with an acidic interior containing a wide variety of hydrolytic enzymes that break down macromolecules such as nucleic acids, proteins, and polysaccharides. Lysosomal storage disorders (LSDs) relate to inherited metabolic disorders that result from defects in lysosomal function resulting in substrate accumulation leading to cellular, tissue, and organ damage, as well as early death. Currently, about 50 distinct LSDs have been identified but a small number of these (fewer than 10) are reported to have treatments.

Gaucher disease is a rare autosomal recessive disorder in which toxic levels of the fatty substance glucocerebroside accumulates in cells. Gaucher disease is caused by low levels of glucocerebrosidase (GCase) activity, an enzyme required to break down glucocerebroside. GCase activity deficiency results in accumulation of glucocerebroside and related compounds in scavenger macrophages/monocytes, forming characteristic Gaucher cells.

Parkinson's disease is a progressive nervous system disorder caused by loss of nerve cells in part of the brain. One of the most common genetic risk factors for Parkinson's and other neurodegenerative disorders is the loss of function or mutations of the GBA1 gene, the gene encoding GCase. Parkinson's disease is characterized by the accumulation of alpha-synuclein, some studies have suggested that enhanced GCase activity resulting in reduction of GCase substrates also results in clearance of pathological alpha-synuclein (see Mazzulli et al., J Neurosci. 2016 Jul. 20; 36 (29): 7693-706. doi: 10.1523/JNEUROSCI.0628-16.2016).

Currently there are no curative therapies for Gaucher or Parkinson's disease. For Gaucher disease, enzyme replacement therapy (ERT) is used to replace the deficient GCase with a substitute (e.g., imiglucerase, velaglucerase alfa), which allows the body to break down the substrates that otherwise build up in the liver and spleen. Current approaches to ERT, however, have drawbacks including, for example, only targeting with mannose receptors (macrophage cells), and tissue penetration (requiring higher dosing), substandard efficacy in bone and lung, long and frequent infusions, immunogenicity, and the like. Therefore, there is a need for safe and effective treatments for Gaucher and Parkinson's disease.

### SUMMARY

The present disclosure is directed to a composition comprising glucocerebrosidase wherein at least 50% of the glucocerebrosidase comprises at least one phosphorylated N-linked oligosaccharide.

In some embodiments at least 25% of the glucocerebrosidase N-linked glycans are bis-phosphorylated. In some embodiments, at least 50% of the glucocerebrosidase N-linked glycans are bis-phosphorylated.

In any of the foregoing embodiments, less than 10% of the glucocerebrosidase N-linked glycans are non-phosphorylated mannose groups.

In any of the foregoing embodiments, less than 50% of the glucocerebrosidase glucocerebrosidase N-linked glycans are complex type N-glycans.

In any of the foregoing embodiments, less than 5% of the glucocerebrosidase glucocerebrosidase N-linked glycans are hybrid type N-glycans.

In any of the foregoing embodiments, the composition comprises less than 5 nanomolar affinity for CIMPR.

In any of the foregoing embodiments, the composition comprises from about 1 to about 50-fold greater activity than imiglucerase.

In any of the foregoing embodiments, the composition comprises from about 1 to about 50-fold greater activity than imiglucerase in at least one tissue selected from the group consisting of bone, bone marrow, spine, brain, spleen, liver, diaphragm, triceps, and quad.

In any of the foregoing embodiments, the composition comprises from about 0.1 to 5-fold greater ability to reduce a substrate compared to imiglucerase.

In any of the foregoing embodiments, the composition comprises from about 0.1 to 5-fold greater ability to reduce a substrate compared to imiglucerase in at least one tissue selected from the group consisting of serum, liver, spleen, bone marrow, bone, lung, diaphragm, quad, spine, and triceps.

In any of the foregoing embodiments, the substrate is selected from the group consisting of is Lyso-GL1, lyso-GB1, C18 glucocerebroside, C24 glucocerebroside, and 1-β-D-Glucosylsphingosine.

In any of the foregoing embodiments, the glucocerebrosidase is capable of being endogenously hydrolyzed.

The present disclosure is also directed to a method of treating Gaucher disease, the method comprising administering to a subject an effective amount of the composition of any of any of the foregoing embodiments.

In some embodiments, administering comprises enzyme replacement therapy.

In some embodiments, administering comprises administration by an (intra) nasal route.

### BRIEF DESCRIPTION OF THE DRAWINGS

For the purpose of illustrating the disclosure, there are depicted in the drawings certain embodiments of the disclosure. However, the disclosure is not limited to the precise arrangements and instrumentalities of the embodiments depicted in the drawings.

FIG. 1A depicts CIMPR affinity chromatography, illustrating the percent of M011 that can interact with the CIMPR via phosphorylated N-linked glycans.

FIG. 1B depicts CIMPR affinity chromatography, illustrating the percent of recombinant wild-type GCase expressed without S1S3 phosphotransferase that can interact with the CIMPR via phosphorylated N-linked glycans.

FIG. 1C depicts CIMPR affinity chromatography illustrating the percent of imiglucerase that can interact with the CIMPR via phosphorylated N-linked glycans.

FIG. 2 depicts CIMPR binding affinity determination of M011 expressed in CHO cells compared to wild-type hGCase expressed without S1S3 phosphotransferase and imiglucerase.

FIG. 3 depicts cellular uptake of M011 and imiglucerase as measured by internalized GCase activity.

FIG. 4 depicts stability of M011 and imiglucerase at pH 7.0.

FIG. 5 depicts aggregation of M011 and imiglucerase at pH 7.0.

FIG. 6A depicts half-life of M011 and imiglucerase in mice at 1.5 mg/kg dosage.

FIG. 6B depicts half-life of M011 and imiglucerase in mice at 0.4 mg/kg dosage.

FIG. 7A depicts activity of M011 and imiglucerase in various mouse tissues after 1.5 mg/kg dosage.

FIG. 7B depicts activity of M011 and imiglucerase in various mouse tissues after 0.4 mg/kg dosage.

FIG. 8 depicts distribution of GCase in liver, spleen, and lung tissue via IHC analysis.

FIG. 9 depicts macrophage levels as detected by anti-CD68 antibody staining in liver and lung tissues of mice treated with M011 or imiglucerase.

FIG. 10 depicts Gaucher Storage cell measurement by H&E staining in liver and lung tissue of mice treated with M011 or imiglucerase.

FIG. 11 depicts levels of C18 and C24 glucosylceramide in liver, spleen, and lung in mice treated with M011 or imiglucerase at 2 and 4 weeks.

FIG. 12 is a flowchart representing treatment and evaluation schedule for a short term 2 and 4-dose efficacy study in a Gaucher mouse model.

FIG. 13 depicts serum 1-β-D-Glucosylsphingosine levels in mice treated with M011 or imiglucerase.

FIG. 14 depicts liver 1-β-D-Glucosylsphingosine levels in mice treated with M011 or imiglucerase.

FIG. 15 depicts spleen 1-β-D-Glucosylsphingosine levels in mice treated with M011 or imiglucerase.

FIG. 16 depicts bone marrow 1-β-D-Glucosylsphingosine levels in mice treated with M011 or imiglucerase.

FIG. 17 depicts bone marrow C24 glucosylceramide levels in mice treated with M011 or imiglucerase.

FIG. 18 depicts bone 1-β-D-Glucosylsphingosine in mice treated with M011 or imiglucerase.

FIG. 19 depicts diaphragm 1-β-D-Glucosylsphingosine in mice treated with M011 or imiglucerase.

FIG. 20 depicts quad 1-β-D-Glucosylsphingosine in mice treated with M011 or imiglucerase.

FIG. 21 depicts GCase activity in bone marrow in mice treated with M011 or imiglucerase.

FIG. 22 depicts 1-β-D-Glucosylsphingosine levels in bone marrow in mice treated with M011 or imiglucerase.

FIG. 23 depicts GCase activity in spine in mice treated with M011 or imiglucerase.

FIG. 24 depicts 1-β-D-Glucosylsphingosine levels in spine in mice treated with M011 or imiglucerase.

FIG. 25 depicts GCase activity in brain in mice treated with M011 or imiglucerase.

FIG. 26 depicts 1-β-D-Glucosylsphingosine levels in brain in mice treated with M011 or imiglucerase.

FIG. 27 depicts GCase activity in spleen in mice treated with M011 or imiglucerase.

FIG. 28 depicts 1-β-D-Glucosylsphingosine levels in spleen in mice treated with M011 or imiglucerase.

FIG. 29 depicts GCase activity in liver in mice treated with M011 or imiglucerase.

FIG. 30 depicts 1-β-D-Glucosylsphingosine levels in liver in mice treated with M011 or imiglucerase.

FIG. 31 depicts GCase activity in diaphragm in mice treated with M011 or imiglucerase.

FIG. 32 depicts 1-β-D-Glucosylsphingosine levels in diaphragm in mice treated with M011 or imiglucerase.

FIG. 33 depicts GCase activity in triceps in mice treated with M011 or imiglucerase.

FIG. 34 depicts 1-β-D-Glucosylsphingosine levels in triceps in mice treated with M011 or imiglucerase.

FIG. 35 depicts GCase activity in quad in mice treated with M011 or imiglucerase.

FIG. 36 depicts 1-β-D-Glucosylsphingosine levels in quad in mice treated with M011 or imiglucerase.

FIG. 37A characterizes a 50 L lot production of M011.

FIG. 37B depicts the glycan profiling for the 50 L M011 lot.

FIG. 37C depicts CIMPR affinity chromatography for the 50 L M011 lot.

FIG. 38A characterizes a 250 L lot production of M011.

FIG. 38B depicts the glycan profiling for the 250 L M011 lot.

FIG. 38C depicts CIMPR affinity chromatography for the 250 L M011 lot.

FIG. 39A depicts the CIMPR affinity profile of M011 expressed in HEK293 cells.

FIG. 39B depicts the CIMPR affinity profile of M011 expressed in a stable clonal pool of CHO cells.

FIG. 39C depicts the CIMPR affinity profile of M011 expressed a final clone of CHO cells.

FIG. 40 depicts the GCase activity in blood serum from mice treated with M011 or imiglucerase.

FIG. 41A depicts M011 and imiglucerase uptake in liver of mice following treatment.

FIG. 41B depicts M011 and imiglucerase uptake in spleen of mice following treatment.

FIG. 41C depicts M011 and imiglucerase uptake in bone marrow of mice following treatment.

FIG. 41D depicts M011 and imiglucerase uptake in brain of mice following treatment.

### DETAILED DESCRIPTION

The current invention is directed to novel compositions comprising GCase having increased phosphorylation of High Mannose N-linked Oligosaccharides and methods of use thereof.

### Definitions

Unless defined otherwise, all technical and scientific terms used herein have the same meaning as commonly understood by one of ordinary skill in the art to which the invention pertains. In describing and claiming the present invention, the following terminology will be used.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of the vims particle or infectious units generated from vector of the invention which is required to prevent the particular disease condition, or which reduces the severity of and/or ameliorates the disease condition or at least one symptom thereof or condition associated therewith.

### Compositions of the Disclosure

The disclosure provides a composition comprising GCase having increased phosphorylation of High Mannose N-linked Oligosaccharides.

Lysosomal proteins, like GAA, are modified by N-glycosylation, which refers to transfer of a GlcNAc2-Man9-Glc3 structure to the Asn-X-Ser/Thr (x any amino acid except His or Pro) motif. These glycoconjugates contain a GlcNAc2 mannose (Man)₃ core. From there, the glycoconjugates are processed. Processing can include, for example, phosphorylation, trimming and rebuilding. The final structure may be classed as a high-mannose N-glycan, a hybrid N-glycan or a complex N-glycan. Over 130 different N-linked glycan structures are possible.

Additionally, the N-glycan may be phosphorylated to contain one or more mannose 6-phosphate (M6P) groups. For example, a N-glycan on a lysosomal protein molecule may bear one M6P group (mono-phosphorylated) or two M6P groups (bis-phosphorylated) on the same N-linked oligosaccharide. The M6P serves as a cellular signal, directing lysosomal proteins to lysosomes—their site of activity—through membrane bound receptors (e.g., cation-independent mannose 6-phosphate receptor or CIMPR). The number of M6P groups affects the binding affinity to CIMPR—a bis-phosphorylated glycan has a higher binding affinity than a mono-phosphorylated glycan.

### M6P Content.

The GCase according to the present disclosure comprises a high percentage of bis-phosphorylated N-linked oligosaccharides. In some embodiments, at least 25% of the N-linked glycans on the GCase are bis-phosphorylated. For example, at least 25%, 30%, 35%, 40%, 45%, 50%, 55%, 60%, or 65% of the N-linked glycans on the GCase are bis-phosphorylated.

The GCase according to the present disclosure comprises a higher percentage of phosphorylated N-linked oligosaccharides and a corresponding lower percentage of non-phosphorylation phosphorylation-linked oligosaccharides. In some embodiments, less than 10% of the N-linked glycans on the GCase are not phosphorylated. For example, less than 10%, 9%, 8%, 7%, 6%, 5%, 4%, 3%, 2%, or 1% of the N-linked glycans on the GCase are non-phosphorylated. In some embodiments, at least 30% of the N-linked glycans on the GCase are phosphorylated. For example, at least 30%, 35%, 40%, 45%, 50%, 55%, 60%, 65%, 70%, 75%, 80%, 85%, 90%, or 95% of the N-linked glycans on the GCase are phosphorylated. In some embodiments, at least 10% of the mannose groups on the GCase are mono-phosphorylated. For example, at least 10%, 15%, 20%, 25%, 30%, or 35% of the mannose groups on the GCase are mono-phosphorylated.

In a preferred embodiment, less than 5% of the N-linked glycans on the GCase are non-phosphorylated and at least 40% of the phosphorylated N-linked glycans on the GCase are bis-phosphorylated.

In some embodiments, on average the N-glycans contain greater than 0.25 mol/mol of bis-M6P. For example, on average the N-glycans contain greater than 0.25 mol/mol, 0.5 mol/mol, 0.75 mol/mol, 1 mol/mol, 1.25 mol/mol, 1.5 mol/mol, 1.75 mol/mol, 2 mol/mol, 2.25 mol/mol, 2.5 mol/mol, 2.75 mol/mol, 3 mol/mol, 3.25 mol/mol, 3.5 mol/mol, 3.75 mol/mol, 4 mol/mol, 4.25 mol/mol, 4.5 mol/mol, 4.75 mol/mol, or 5 mol/mol of bis-phosphorylated N-linked glycans/mol protein. In a preferred embodiment, the GCase according to the present disclosure comprises on at least 2 moles of bis-M6P per mole of GCase

### Glycan Type.

The GCase according to the present disclosure comprises a low level of neutral glycans. In some embodiments, the GCase comprises less than 5% neutral glycans, which provides reduced rates of non-productive clearance via mannose receptors and asialoglycoprotein receptors and improved ability to target cells.

The GCase according to the present disclosure comprises a reduced percentage of complex and hybrid-type N-glycans. In some embodiments, less than 50% of the total N-glycans on the GCase are complex type N-glycans. For example, less than 50%, 45%, 40%, 35%, 30%, 25%, 20%, 15%, 10%, or 5% of the total N-glycans on the GCase are complex type N-glycans. In some embodiments, less than 5% of the total N-glycans on the GCase are hybrid type N-glycans. For example, less than 50%, 45%, 40%, 35%, 30%, 25%, 20%, 15%, 10%, 5%, 4%, 3%, 2%, or 1% of the total N-glycans on the GCase are hybrid type N-glycans. In a preferred embodiment, less than 20% of the total N-glycans on the GCase are complex type N-glycans and less than 1% of the total N-glycans on the GCase are hybrid type N-glycans.

### Glycosylation Profile

The wild-type sequence of GCase has five N-glycosylation sites. A modified sequence of GCase comprises six N-glycosylation sites. The present disclosure provides for an improved glycosylation profile GCase derived from either the modified or wild-type sequence of GCase.

In some embodiments, at least 10% of GCase is phosphorylated at the first N-glycosylation site. For example, at least 10%, 15%, 20%, 25%, 30%, 35%, 40%, 45%, 50%, 55%, 60%, 65%, 70%, 75%, 80%, 85%, 90%, or 95% of the GCase can be phosphorylated at the first N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GCase bears a mono-M6P unit at the first N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GCase bears a bis-M6P unit at the first N-glycosylation site.

In some embodiments, at least 10% of GCase is phosphorylated at the second N-glycosylation site. For example, at least 10%, 15%, 20%, 25%, 30%, 35%, 40%, 45%, 50%, 55%, 60%, 65%, 70%, 75%, 80%, 85%, 90%, or 95% of the GCase can be phosphorylated at the second N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GCase bears a mono-M6P unit at the second N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GCase bears a bis-M6P unit at the second N-glycosylation site.

In some embodiments, at least 10% of GCase is phosphorylated at the third N-glycosylation site. For example, at least 10%, 15%, 20%, 25%, 30%, 35%, 40%, 45%, 50%, 55%, 60%, 65%, 70%, 75%, 80%, 85%, 90%, or 95% of the GCase can be phosphorylated at the third N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GCase bears a mono-M6P unit at the third N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GCase bears a bis-M6P unit at the third N-glycosylation site.

In some embodiments, at least 10% of GCase is phosphorylated at the fourth N-glycosylation site. For example, at least 10%, 15%, 20%, 25%, 30%, 35%, 40%, 45%, 50%, 55%, 60%, 65%, 70%, 75%, 80%, 85%, 90%, or 95% of the GCase can be phosphorylated at the fourth N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GCase bears a mono-M6P unit at the fourth N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GCase bears a bis-M6P unit at the fourth N-glycosylation site.

In some embodiments, at least 10% of GCase is phosphorylated at the fifth N-glycosylation site. For example, at least 10%, 15%, 20%, 25%, 30%, 35%, 40%, 45%, 50%, 55%, 60%, 65%, 70%, 75%, 80%, 85%, 90%, or 95% of the GCase can be phosphorylated at the fifth N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GCase bears a mono-M6P unit at the fifth N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GCase bears a bis-M6P unit at the fifth N-glycosylation site.

In some embodiments, at least 10% of GCase is phosphorylated at the sixth N-glycosylation site. For example, at least 10%, 15%, 20%, 25%, 30%, 35%, 40%, 45%, 50%, 55%, 60%, 65%, 70%, 75%, 80%, 85%, 90%, or 95% of the GCase can be phosphorylated at the sixth N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GCase bears a mono-M6P unit at the sixth N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GCase bears a bis-M6P unit at the sixth N-glycosylation site.

### CIMPR Binding and Cell Uptake.

The GCase according to the present disclosure comprises increased cation independent mannose 6-phosphate receptor (CIMPR) binding and cellular uptake due to increased phosphorylation. In some embodiments, the GCase has less than 5 nanomolar affinity for the CIMPR. For example, the GCase has less than 5 nanomolar, 4 nanomolar, 3 nanomolar, 2 nanomolar, or 1 nanomolar for the CIMPR.

In some embodiments, at least 30% of the GCase can interact with the CIMPR. For example, at least 30%, 35%, 40%, 45%, 50%, 55%, 60%, 65%, 70%, 75%, 80%, 85%, 90%, or 95% of the GCase can interact with the CIMPR.

In a preferred embodiment, the GCase has less than 4 nanomolar affinity for the CIMPR and at least 75% of the GCase can interact with CIMPR.

### Increased GCase Activity.

In some embodiments, the GCase of the present invention has more GCase activity compared to imiglucerase. In some embodiments, activity is measured by the ability to hydrolyze the synthetic 4MU-beta-Glc substrate to 4MU and Glc. For example, the GCase of the present invention may have at least 1 to 50-fold greater activity compared to imiglucerase.

In some embodiments, the GCase of the present invention has more GCase activity compared to imiglucerase, likely due to greater stability and better targeting of the GCase into the cell, in at least one of bone, bone marrow, spine, brain, spleen, liver, diaphragm, triceps, or quad tissues.

### Ability to Reduce GC Substrate.

In some embodiments, the GCase of the present invention is more effective at reducing substrate compared to imiglucerase. For example, the GCase of the present invention may be about 0.1 to 5-fold greater reduction of substrate compared to imiglucerase.

In some embodiments, the GCase of the present invention is more effective at reducing substrate compared to imiglucerase in at least one of serum, liver, spleen, bone marrow, bone, lung, diaphragm, quad, spine, and triceps tissues.

In any of the foregoing embodiments, the substrate more effectively reduced by the GCase of the present invention compared to imiglucerase is Lyso-GL1, lyso-GB1, C18 glucocerebroside, C24 glucocerebroside, or 1-β-D-Glucosylsphingosine.

Longer Half-Life. The GCase of the present disclosure comprises a longer activity half-life compared to imiglucerase, likely due to either clearance by the mannose receptor for imiglucerase versus clearance by the CIMPR receptor for GCase of the present disclosure, and/or the greater stability of the GCase of the present disclosure at neutral pH.

Enzymatic Phosphorylation. Phosphorylation of the GCase of the present disclosure is achieved enzymatically, not chemically, so the GCase is capable of being degraded endogenously in the lysosome. A non-limiting example of compounds and compositions that can be endogenously hydrolyzed include compounds and compositions that lack a hydrazone bond. Unlike unnatural chemical bonds like hydrazone bonds that are typically used for chemical conjugation of synthetic glycans, the GCase of the present disclosure comprises natural glycosidic bonds that can be properly processed in lysosomes after cellular uptake in target cells.

### Pharmaceutical Compositions and Formulations.

Also provided herein is a pharmaceutical composition comprising GCase of the disclosure.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. In some embodiments, the presently disclosed compositions can be formulated in a natural capsid, a modified capsid, as a naked RNA, or encapsulated in a protective coat.

The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. In some embodiments, the disclosure provides a pharmaceutical composition for treating a subject suffering from Gaucher disease, Parkinson's disease, or another neurodegenerative disease linked to mutations or loss of function in GBA1.

Pharmaceutically acceptable carriers, which are useful, include, but are not limited to, glycerol, water, saline, ethanol and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The disclosed composition may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. In some embodiments, the preservative is a combination of about 0.5% to 2.0% benzyl alcohol and 0.05% to 0.5% sorbic acid.

The composition may include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. In some embodiments, BHT and disodium edetate are the antioxidant and the chelating agent respectively for some compounds, however, other suitable and equivalent antioxidants and chelating agents may be substituted therefore as would be known to those skilled in the art.

The disclosure provides a pharmaceutical composition comprising GCase of the disclosure and a pharmaceutically acceptable carrier.

The compositions of the disclosure may be used for enzyme replacement therapy (ERT). Alternatively or in addition, the compositions of the disclosure may be used for gene therapy.

### Methods of the Disclosure

The disclosure provides method to treat a subject deficient for GCase with Gaucher disease, Parkinson's disease, or another neurodegenerative disease linked to mutations or loss of function in GBA1, or in a subject at risk for developing Gaucher disease, Parkinson's disease, or another neurodegenerative disease linked to mutations or loss of function in GBA1. The method introduces phosphorylated GCase thereby treating the subject or preventing the occurrence of disease in the subject. Further, the method improves quality of life in a patient.

The disclosure provides a method of treating Gaucher disease, Parkinson's disease, or another neurodegenerative disease linked to mutations or loss of function in GBA1, the method comprising administering to a subject a composition of the disclosure, thereby treating the disease.

The disclosure provides a method of treating Gaucher disease, Parkinson's disease, or another neurodegenerative disease linked to mutations or loss of function in GBA1, the method comprising administering to a subject a therapeutically effective amount of a composition of the disclosure, thereby treating the disease.

The disclosure provides a method of treating a subject suffering from Gaucher disease, Parkinson's disease, or another neurodegenerative disease linked to mutations or loss of function in GBA1, the method comprising administering to the subject a pharmaceutical composition of the disclosure, treating the subject.

The disclosure provides a method of preventing the occurrence of Gaucher disease, Parkinson's disease, or another neurodegenerative disease linked to mutations or loss of function in GBA1, in a subject in need thereof, the method comprising administering to the subject a pharmaceutical composition of the disclosure, thereby preventing the occurrence of the disease in the subject.

### Administration/Dosing.

The regimen of administration may affect what constitutes an effective amount. For example, the therapeutic formulations may be administered to the patient subject either prior to or after a surgical intervention related to Gaucher disease, Parkinson's disease, or another neurodegenerative disease linked to mutations or loss of function in GBA1, or shortly after the patient was diagnosed with the disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient subject, preferably a mammal, more preferably a human, may be carried out using known procedures, at dosages and for periods of time effective to treat Gaucher disease, Parkinson's disease, or another neurodegenerative disease linked to mutations or loss of function in GBA1 in the subject. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 and 50 mg/kg of body weight/per day.

The composition can be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of Gaucher disease, Parkinson's disease, or another neurodegenerative disease linked to mutations or loss of function in GBA1.

### Routes of Administration.

One skilled in the art will recognize that although more than one route can be used for administration, a particular route can provide a more immediate and more effective reaction than another route.

Routes of administration of the disclosed compositions include inhalational, oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, intra-cisterna magna (ICM), intraspinal, intraventricular, intracerebroventricular, subcutaneous, intramuscular, intradermal, intra-arterial, intravenous, intrabronchial, inhalation, topical administration, implantable ports, or other medical devices for controlled dosing of GCase. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein. In one embodiment, the treatment of Gaucher disease comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal and any combination thereof.

Furthermore, the actual dose and schedule can vary depending on whether the compositions are administered in combination with other pharmaceutical compositions, or depending on interindividual differences in pharmacokinetics, drug disposition, and metabolism. One skilled in the art can easily make any necessary adjustments in accordance with the exigencies of the particular situation.

### EXAMPLES

The disclosure is described with reference to the following Examples. These Examples are provided for the purpose of illustration only and the disclosure should in no way be construed as being limited to these Examples, but rather should be construed to encompass any and all variations which become evident as a result of the disclosure provided herein.

Without further description, it is believed that one of ordinary skill in the art can, using the preceding description and the following illustrative examples, make and utilize the compounds of the present disclosure and practice the claimed methods. The following working examples are not to be construed as limiting in any way the present disclosure.

### Example 1: M6PT Co-Expression Platform to Produce Phosphorylated GCase

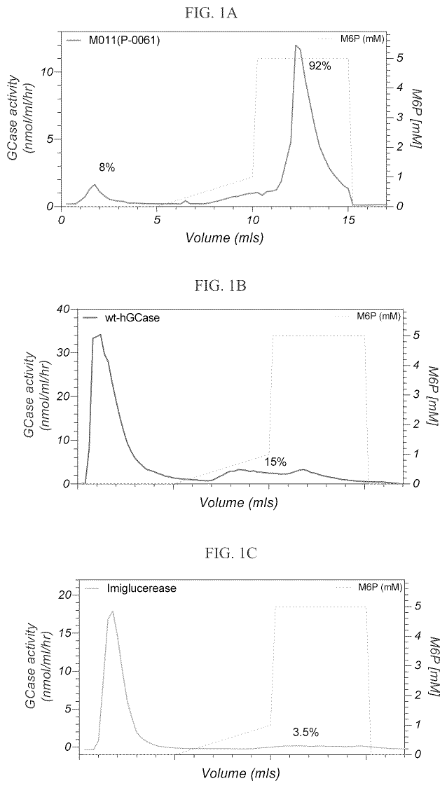

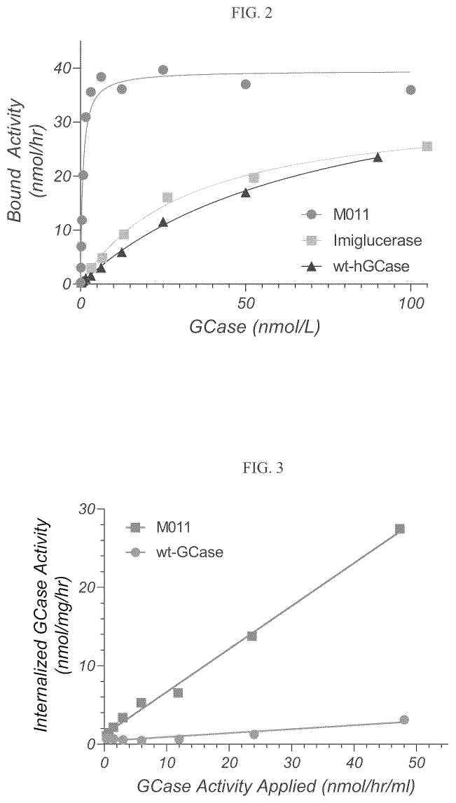

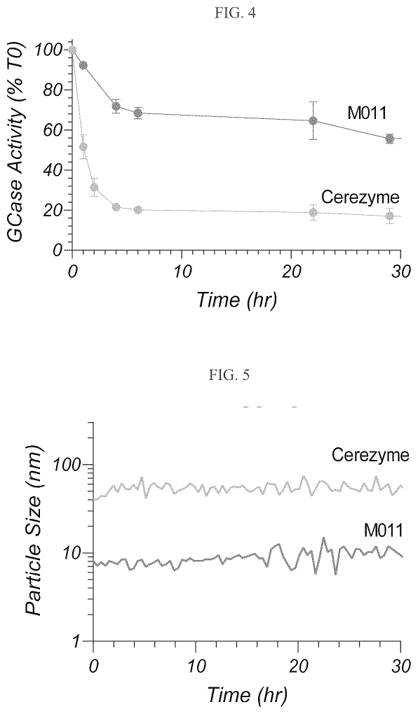

GCase was transiently co-expressed in HEK293 cells utilizing a bicistronic vector encoding the S1S3 variant of N-acetylglucosamine-1-phosphate transferase (PTase) (see Lin Liu et al. Engineering of GlcNAc-1-Phosphotransferase for Production of Highly Phosphorylated Lysosomal Enzymes for Enzyme Replacement Therapy, Mol Ther Methods Clin Dev. 2017 Jun. 16; 5:59-65) and a natural variant of GCase (characterized by Asn instead of Lys at residue 321 (numbering based on GCase lacking 39 a.a. signal sequence)) with reported greater stability at neutral and slightly alkali conditions (M011). M011 (FIG. 1A) exhibits higher amount of GCase that can bind the CIMPR compared to wild-type human GCase expressed in HEK293 cells (FIG. 1B) and standard of care Cerezyme/imiglucerase expressed in CHO cells that were treated with exo-glycosidases to expose core mannose residues for mannose receptor binding and delivery (FIG. 1C). Moreover, as depicted in FIG. 2, M011 exhibits greater cellular uptake compared to wild-type human GCase expressed in HEK293 cells and standard of care Cerezyme/imiglucerase expressed in CHO cells that were treated with exo-glycosidases to expose core mannose residues for mannose receptor binding and delivery. Compared to wild-type human GCase, M011 exhibits increased cellular uptake as measured by internalized GCase activity (FIG. 3). And compared to Cerezyme, M011 exhibits greater stability at pH 7.0 (FIG. 4), and less prone to aggregate at pH 7.0 (see FIG. 5).

### Example 2: Single Dose PK/PD Study in D409V/Null Gaucher Mouse Model

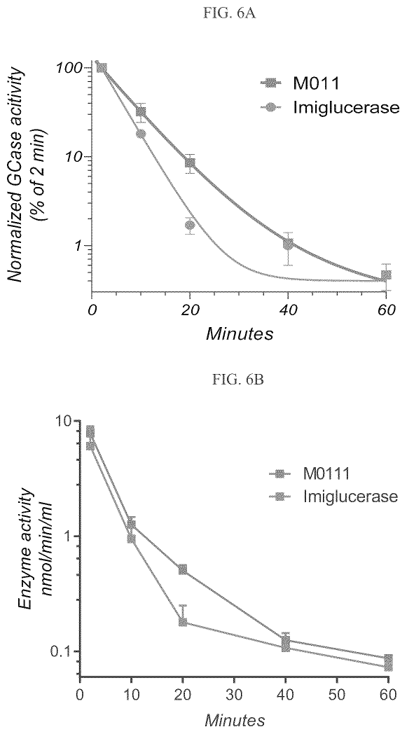

A PK/PD study in the D409V/null Gaucher mouse model was performed using the Gaucher disease standard of care, Cerezyme/imiglucerase, and purified M011 (as described in Example 1). Briefly, 3 animals received a tail vein injection of either about 1.5 mg/kg or 0.4 mg/kg of GCase. For the serum or plasma pharmacokinetic data, plasma samples were collected at 2, 10, 20, 40 and 60 mins. Activity was measured using a synthetic substrate, 4-methylumbelliferyl-beta-D-glucopyranoside (4MU-β-Glc). As can be seen in FIG. 6A-6B, M011 at both 1.5 mg/kg (FIG. 6A, normalized in the individual animals by setting the 2 min timepoint as 100% activity and subsequent timepoints are a percent of the t=2 min time point) and 0.4 mg/kg (FIG. 6B) appears to have a longer half-life compared to imiglucerase. This longer half-life is a combination of the enzyme being stable and the different clearance pathways.

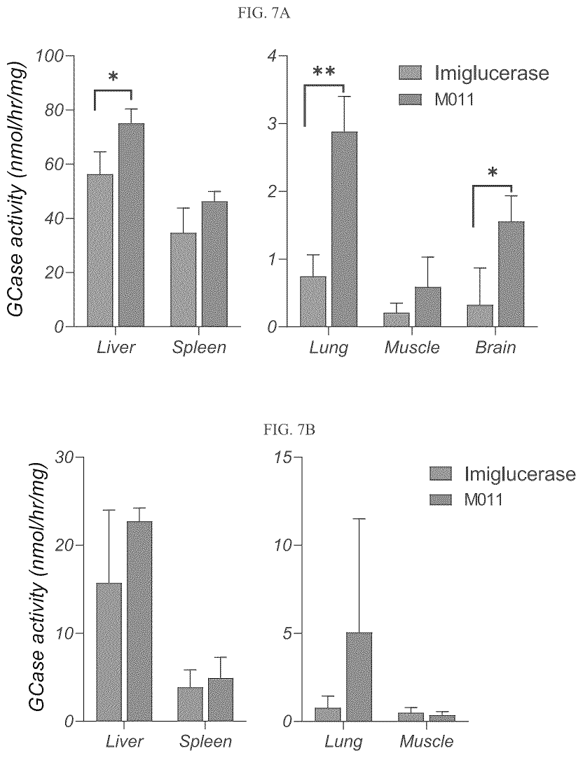

To determine how much GCase was taken up by the tissue, 2 hrs after enzyme injection, tissue was recovered, homogenized and activity measured using the 4MU-beta-Glc substrate. The activity was normalized to total protein in the homogenate as determined by the BCA method for protein determination, a widely used colorimetric method used to measure total protein concentration in cell lysates and tissue homogenates. As shown in FIG. 6A-B, in all tissues evaluated at both 1.5 mg/kg (FIG. 7A) and 0.4 mg/kg (FIG. 7B) there is more activity found in M011 compared to imiglucerase. This is most dramatic in the lung, muscle and brain where imiglucerase has limited activity. When the tissue and sera data is taken together, the advantage of a more stable GCase with greater phosphorylation is apparent for delivering more enzyme to affected tissue. This is the first time that significant amount of GCase has been delivered to the lung, muscle, and heart at these doses.

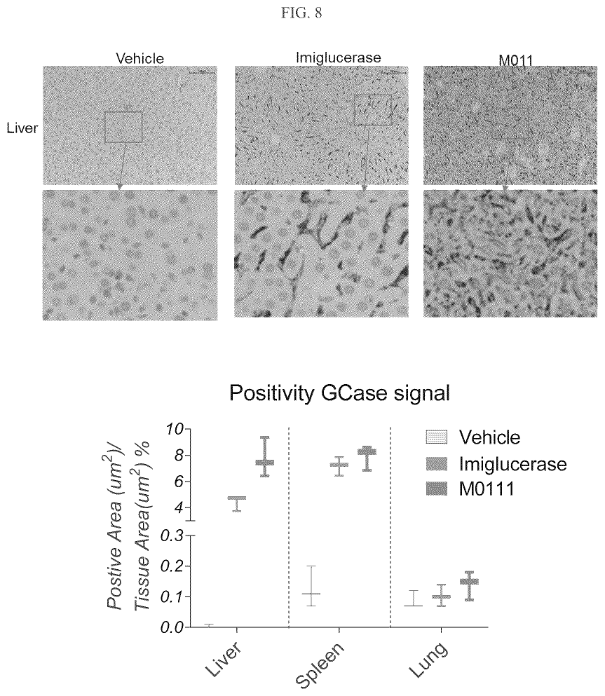

Further, as seen in FIG. 8, IHC analysis of liver, spleen, and lung tissue demonstrates that M011 has broad distribution in cells compared to imiglucerase.

### Example 3: Four Week Study in D409V/Null Gaucher Mouse Model

An efficacy study in the D409V/null Gaucher mouse model was performed using the Gaucher disease standard of care, Cerezyme/imiglucerase, and purified M011.

20 weeks old Gaucher mice were treated with about 1.3-1.5 mg/kg GCase, either M011 or imiglucerase, weekly for four weeks. Seven days later, the tissue of liver and lung was harvested and fixed in 4% paraformaldehyde-PBS, pH 7.4 for immunohistochemistry with CD68 antibody and hematoxylin and eosin (H&E) staining.

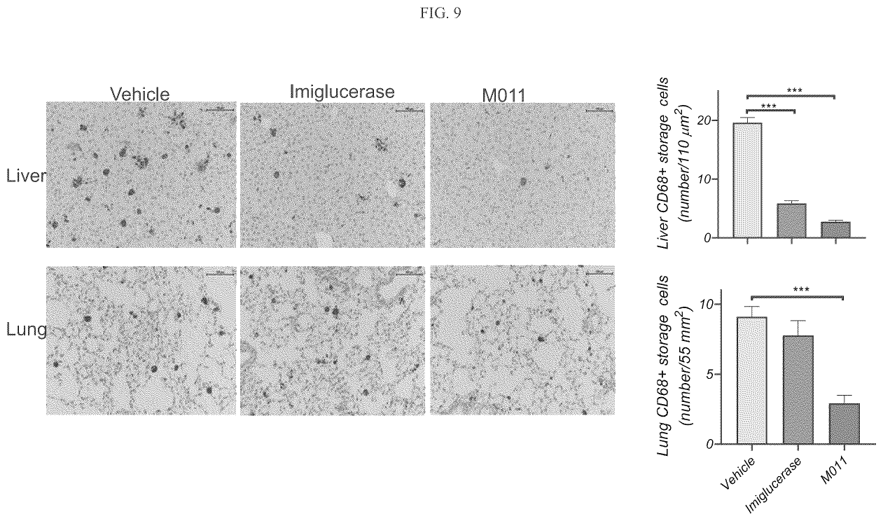

As shown in FIG. 9, M011 has greater efficacy compared to imiglucerase as evidenced by the reduction of macrophage storage in cells in affected tissues as visualized by CD68 antibody reactivity in liver and lung tissues.

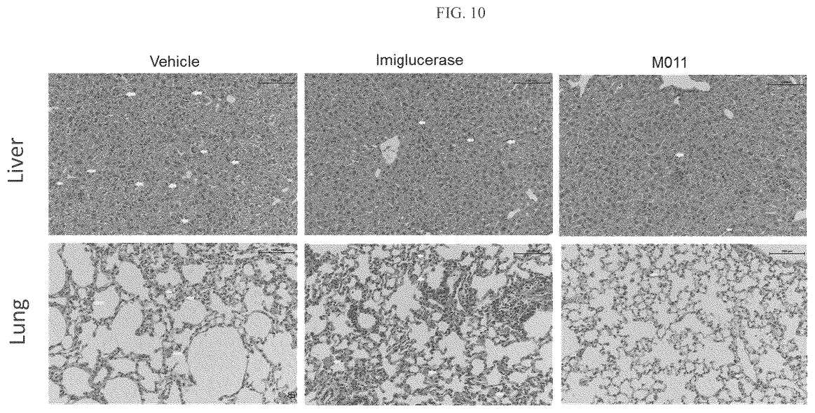

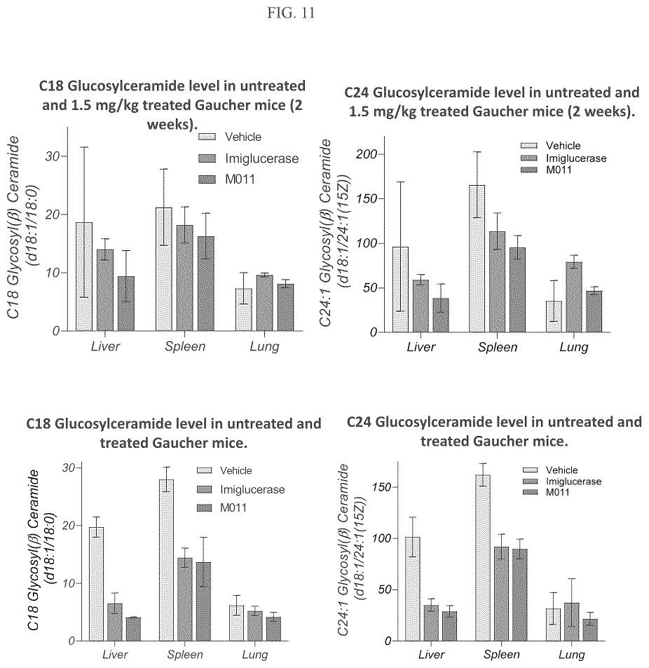

Moreover, as shown in FIG. 10, M011 has greater efficacy compared to imiglucerase as evidenced by the reduction of storage cells in affected tissue as visualized by H&E staining in liver and lung tissue.

In addition to the IHC and H&E analysis, tissue samples were collected at 2 and 4 weeks and homogenized for glucosylceramide analysis. Accumulation of glucosylceramides in the lung is an unmet need of the current standard of care The accumulation of GCase's natural substrate, glucocerebroside was determined in tissue homogenates according to the following steps: 1) An aliquot of 20 μL sample, calibration standard, quality control, dilute quality control, single blank and double blank samples were added to the 96-well plate; 2) Each sample (except the double blank) was quenched with 200 μL IS respectively (double blank sample was quenched with 200 μL Methanol/ACN/H₂O (v:v:v=85:10:5)), and then the mixture was vortex-mixed for 5 min at 800 rpm and centrifuged for 15 min at 3220 g (4000 rpm), 4° C.; 3) 50 μL supernatant was dried with nitrogen and resuspended with Methanol/ACN/H2O (v:v:v=85:10:5), and then fully vortexed for 5 min, the diluted solution were directly injected for LC-MS/MS analysis.

As shown in FIG. 11, at both 2 week and 4 week time points, the animals treated with M011 had lower levels of C18 and C24 glucosylceramide in liver, spleen, and lung compared to animals treated with imiglucerase.

### Example 4: Short Term 2&4 Dose Efficacy Study in D409V/Null Gaucher Mouse Model

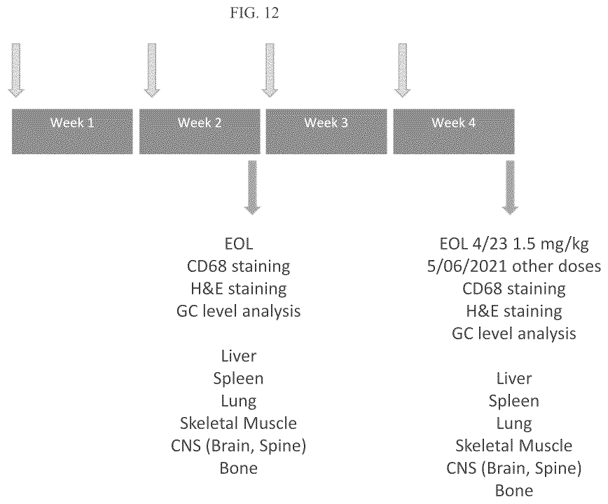

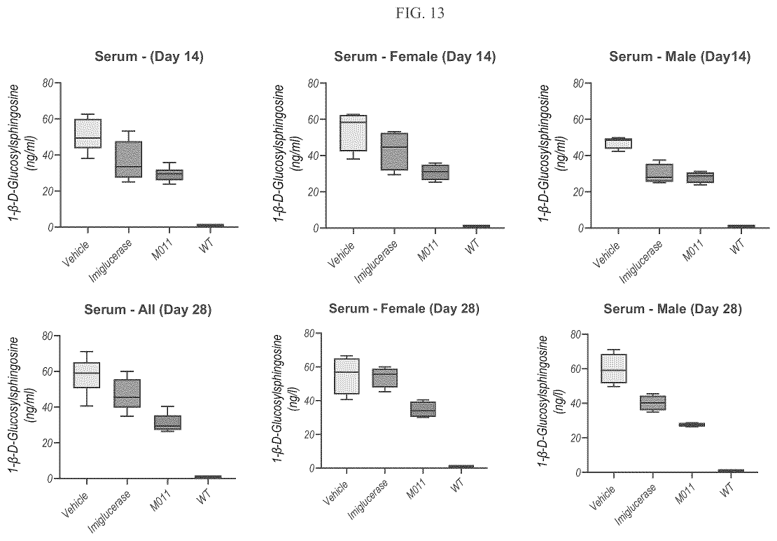

A comparison study in the D409V/null Gaucher mouse model was performed using the Gaucher disease standard of care, Cerezyme/imiglucerase, wild-type rhGCase expressed in the presence of S1S3 PTase produced from a stable cell line from WuXi Biologics. Briefly, 12-14 week old mixed-gender D409V/null mice were dosed weekly with 1.5, 0.75, and 0.3 mg/kg of either imiglucerase, wild-type GCase, or M011. After 2 and 4 weeks, tissue was harvested for IHC with CD68 (data not shown), H&E staining (data not shown), and glucosylceramide level analysis (see FIG. 12).

### Substrate Level Analysis.

Serum. As shown in FIG. 13, at both time points M011 is significantly better at reducing serum 1-β-D-Glucosylsphingosine at 1.5 mg/kg dosage compared to imiglucerase.

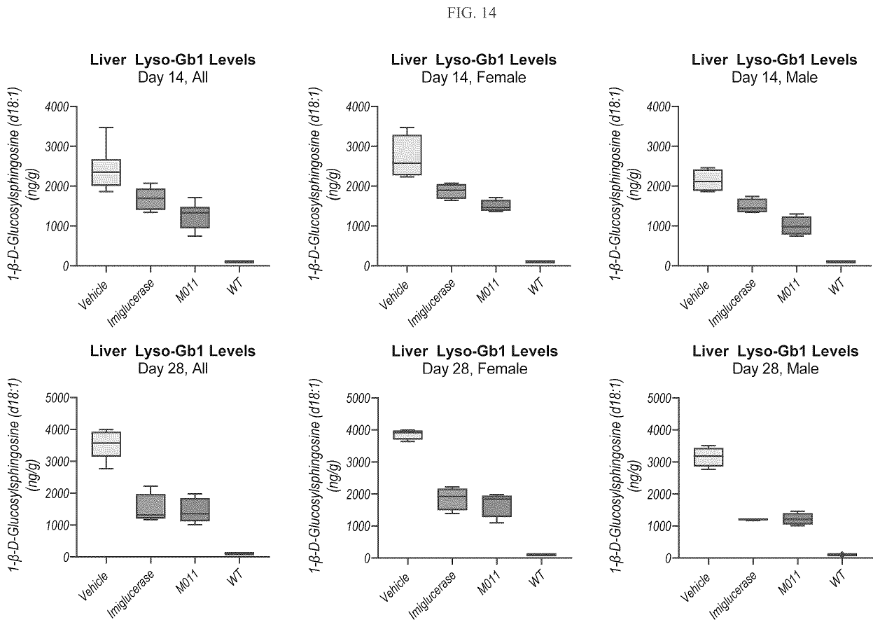

Liver. As shown in FIG. 14, at the two-week time point M011 is significantly better at reducing liver 1-β-D-Glucosylsphingosine compared to imiglucerase, and at the four-week time point M011 and imiglucerase appear to have similar activity in reducing liver Lyso-GL3.

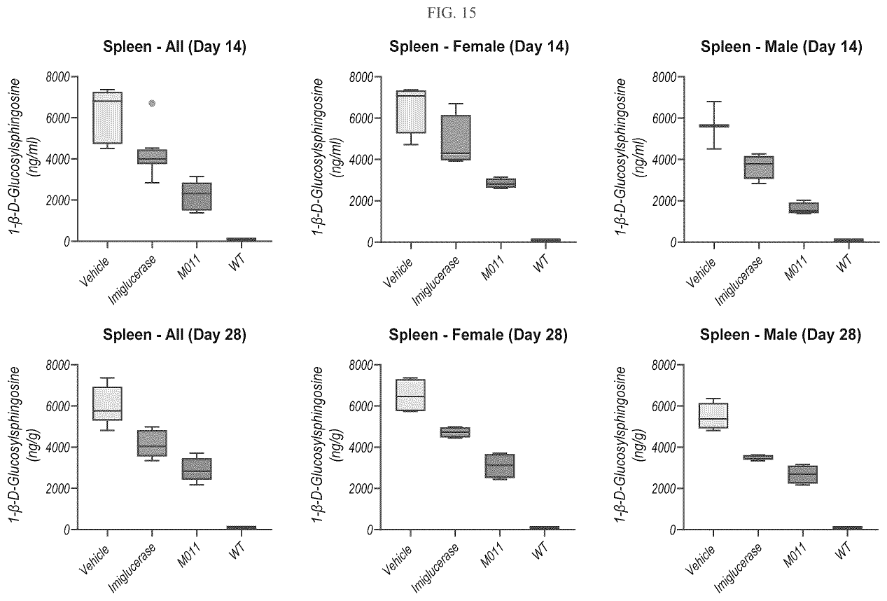

Spleen. As shown in FIG. 15, at both time points M011 is significantly better at reducing spleen 1-β-D-Glucosylsphingosine at 1.5 mg/kg dosage compared to imiglucerase.

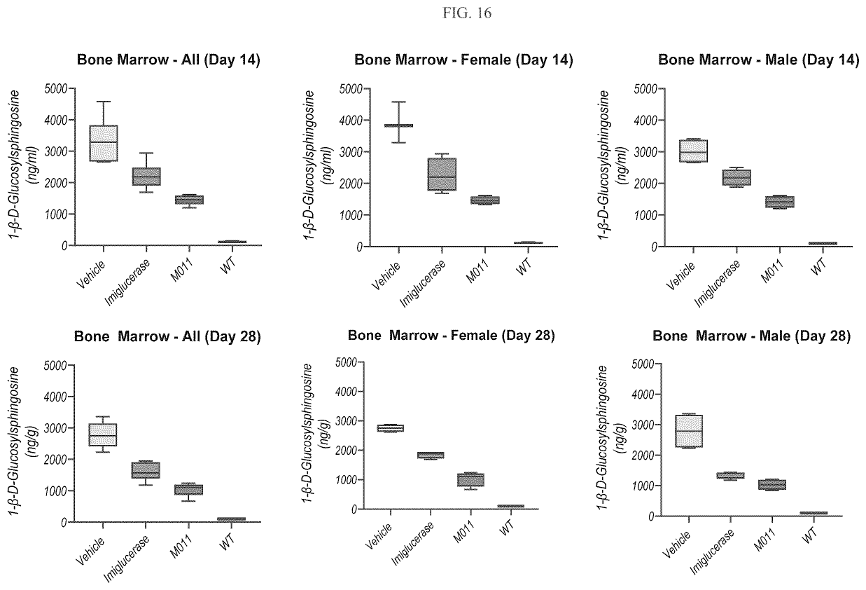

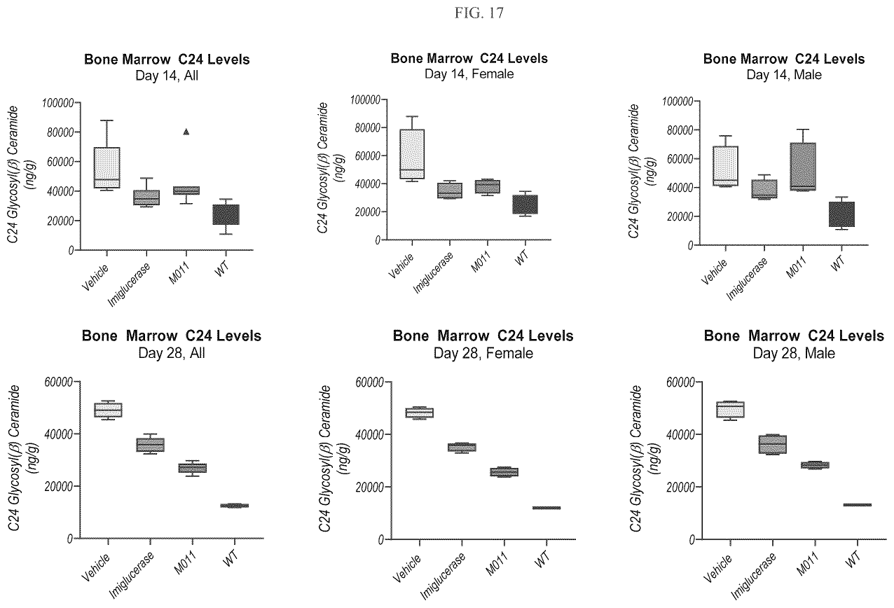

Bone Marrow. As shown in FIG. 16, at both time points M011 is significantly better at reducing bone marrow 1-β-D-Glucosylsphingosine at 1.5 mg/kg dosage compared to imiglucerase. As shown in FIG. 17, the four-week time point shows that M011 is significantly better at reducing bone marrow C24 glucosylceramide levels compared to imiglucerase.

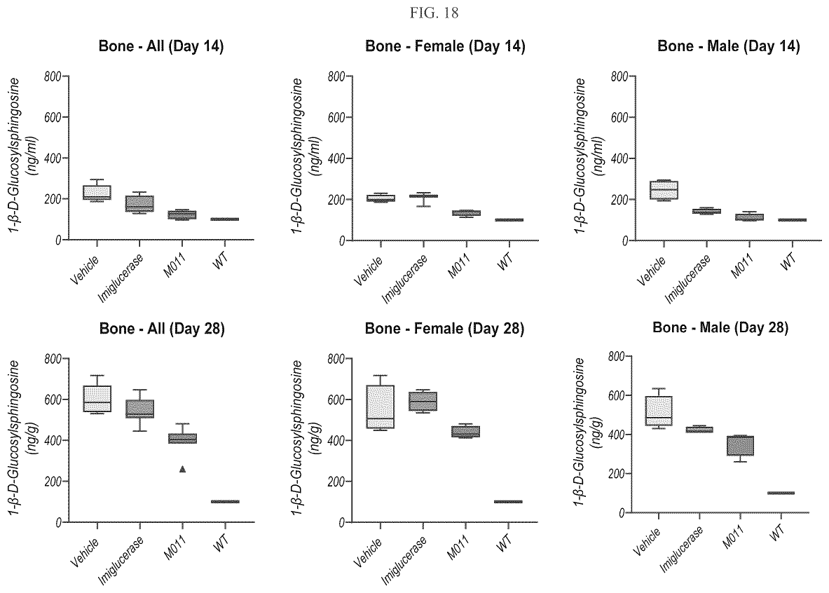

Bone. As shown in FIG. 18, at both time points M011 is significantly better at reducing bone marrow 1-β-D-Glucosylsphingosine at 1.5 mg/kg compared to imiglucerase.

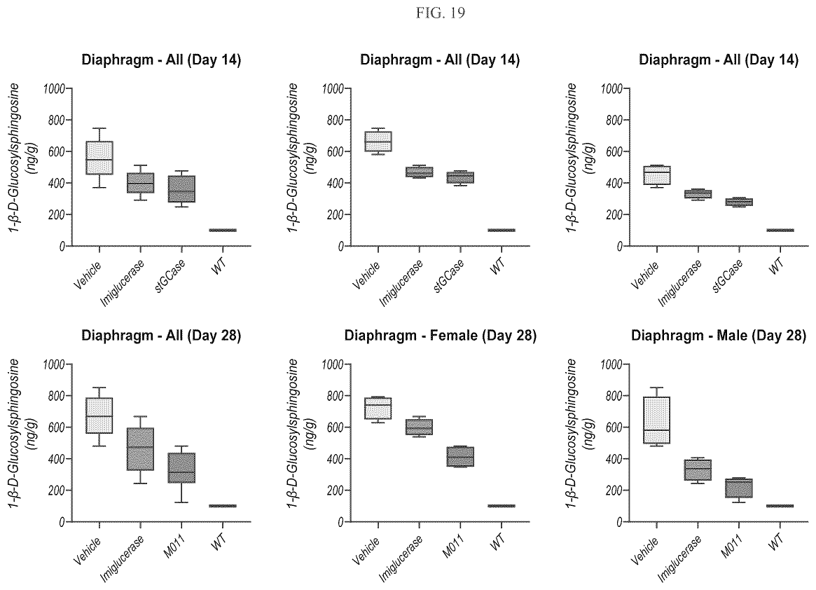

Diaphragm. As shown in FIG. 19, at the four-week time point M011 is significantly better at reducing diaphragm 1-β-D-Glucosylsphingosine at 1.5 mg/kg dosage compared to imiglucerase.

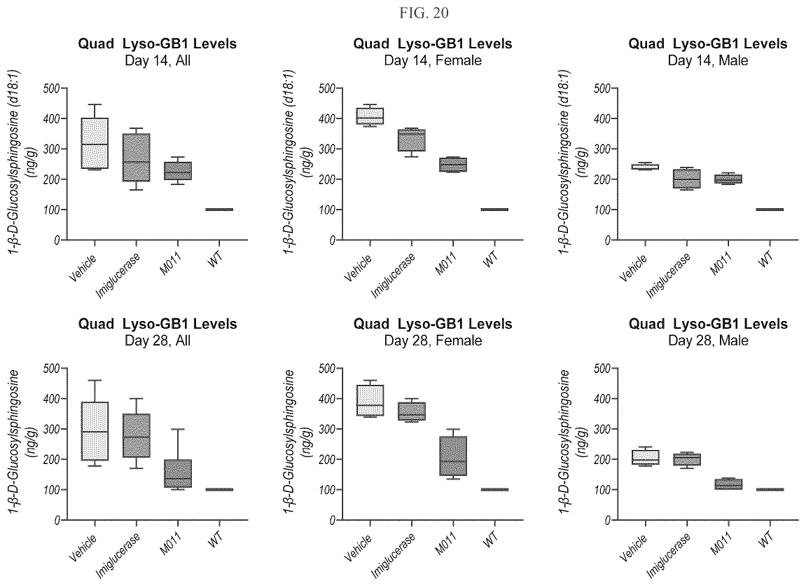

Quad. As shown in FIG. 20, at the four-week time point, M011 is significantly better at reducing quad 1-β-D-Glucosylsphingosine levels at 1.5 mg/kg dosage compared to imiglucerase.

### Example 5: Single Dose PD Study in D409V/Null Gaucher Mouse Model

A dose range study in the D409V/null Gaucher mouse model was performed using M011 produced from a stable cell line from WuXi Biologics. Briefly, D409V/null mice were dosed once with 0.75, 1.5, 3.0, 6.0, or 10 mg/kg M011, or 3.0 mg/kg imiglucerase. One day following dosage, analysis was conducted on activity and GC level analysis.

### GCase Activity and GC Level Analysis.

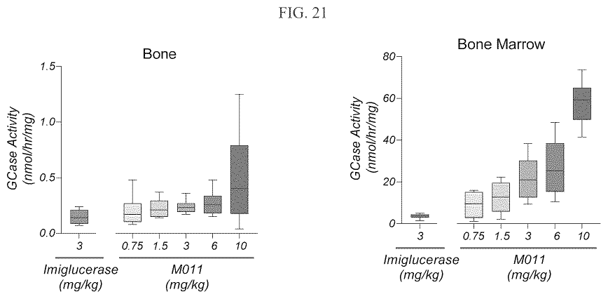

Bone and Bone Marrow. As shown in FIG. 21, greater GCase activity in bone and bone marrow is observed in mice treated with M011 compared to imiglucerase. As shown in FIG. 22, reduced 1-β-D-Glucosylsphingosine in bone and bone marrow is observed in mice treated with M011 compared to imiglucerase, with a dosage-dependent effect for M011 observed (higher concentration M011, more substrate reduction).

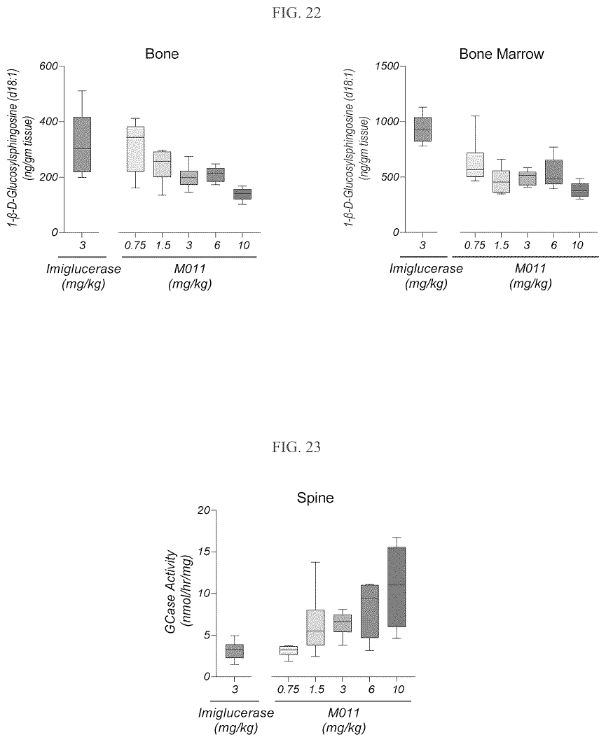

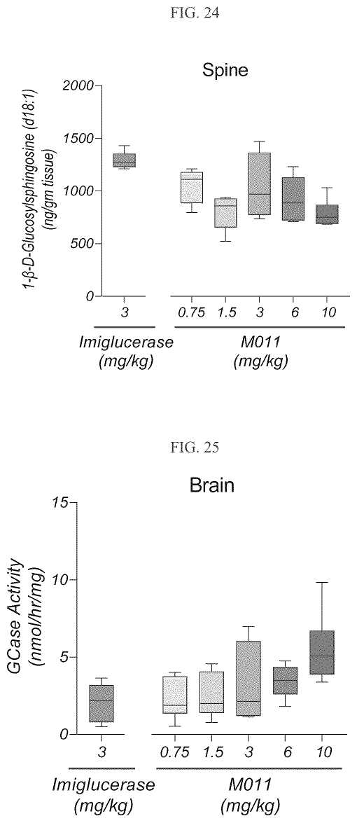

Spine. As shown in FIG. 23, greater GCase activity in spine is observed in mice treated with M011 compared to imiglucerase. As shown in FIG. 24, reduced 1-β-D-Glucosylsphingosine in the spine is observed in mice treated with M011 compared to imiglucerase, with a dosage-dependent effect for M011 observed.

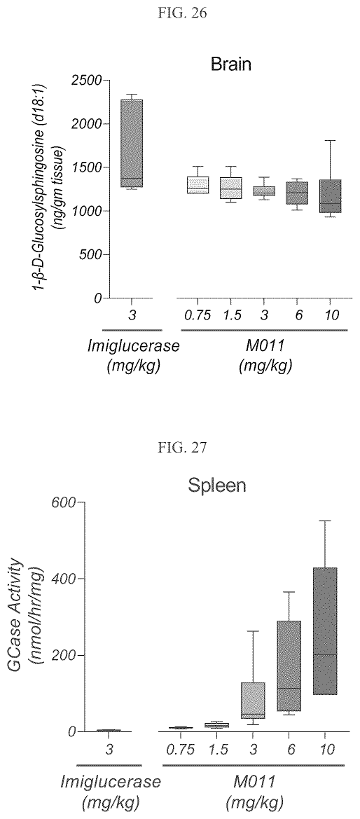

Brain. As shown in FIG. 25, seemingly greater GCase activity in brain is observed in mice treated with M011 compared to imiglucerase. As shown in FIG. 26, results inconclusive regarding the levels of 1-β-D-Glucosylsphingosine, further evaluation needed.

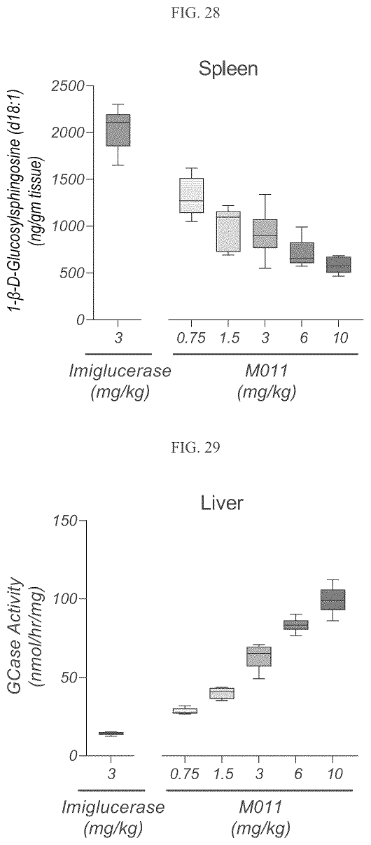

Spleen. As shown in FIG. 27, greater GCase activity in spleen is observed in mice treated with M011 compared to imiglucerase. As shown in FIG. 28, reduced 1-β-D-Glucosylsphingosine in the spleen is observed in mice treated with M011 compared to imiglucerase, with a dosage-dependent effect for M011 observed.

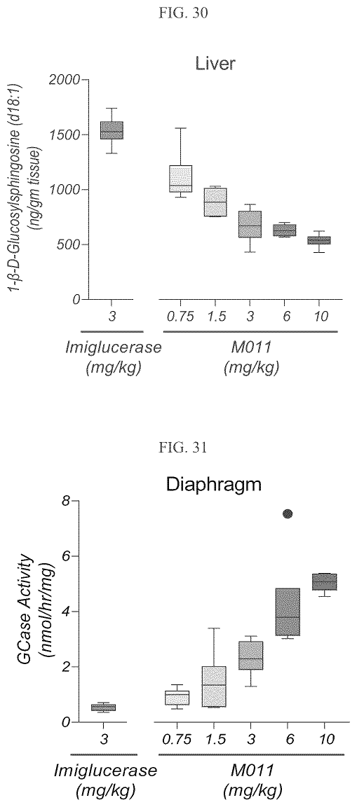

Liver. As shown in FIG. 29, greater GCase activity in liver is observed in mice treated with M011 compared to imiglucerase. As shown in FIG. 30, reduced 1-β-D-Glucosylsphingosine in the liver is observed in mice treated with M011 compared to imiglucerase, with a dosage-dependent effect for M011 observed.

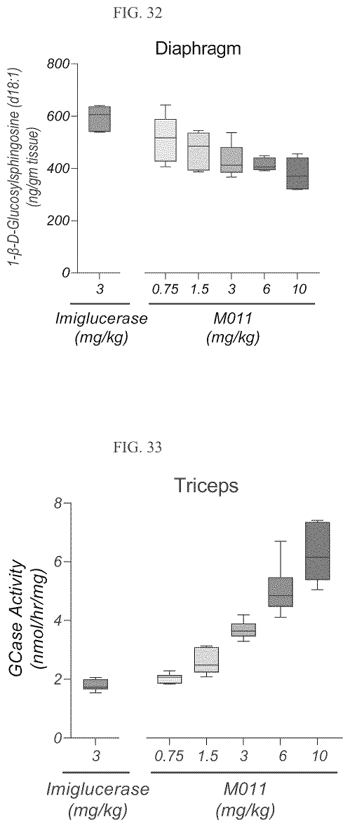

Diaphragm. As shown in FIG. 31, greater GCase activity in diaphragm is observed in mice treated with M011 compared to imiglucerase. As shown in FIG. 32, reduced 1-β-D-Glucosylsphingosine in the diaphragm is observed in mice treated with M011 compared to imiglucerase, with a dosage-dependent effect for M011 observed.

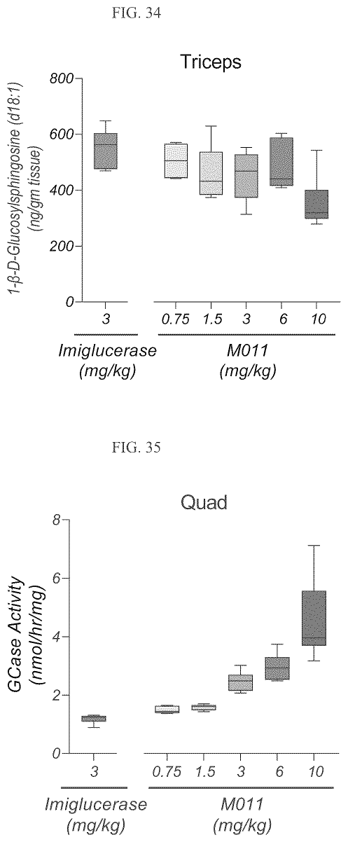

Triceps. As shown in FIG. 33, greater GCase activity in triceps is observed in mice treated with M011 compared to imiglucerase. As shown in FIG. 34, it appears 1-β-D-Glucosylsphingosine in the liver is observed in mice treated with M011 compared to imiglucerase, inconclusive regarding the levels of 1-β-D-Glucosylsphingosine, further evaluation needed.

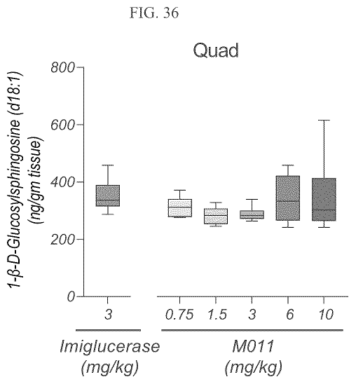

Quad. As shown in FIG. 35, greater GCase activity in quad is observed in mice treated with M011 compared to imiglucerase. As shown in FIG. 36, results inconclusive regarding the levels of 1-β-D-Glucosylsphingosine in the quad, further evaluation needed.

### Example 6: Production Lots of M011 and Glycan Profiling

50 L and 250 L production lots of M011 were made from a final stable CHO clone. Material was expressed in perfusion bioreactors and purified by multiple chromatographic steps.

For glycan profiling, GCase was reduced and alkylated to form a linear protein. The protein was then be digested with peptide-N-glycanase to release all N-linked oligosaccharides. These released glycans were purified and labeled stoichiometrically with a fluorescent probe and separated by rpHPLC according to charge/size and elution monitored by fluorescence. The peaks were be identified by LC-MS/MS and relative area of each glycan form determined.

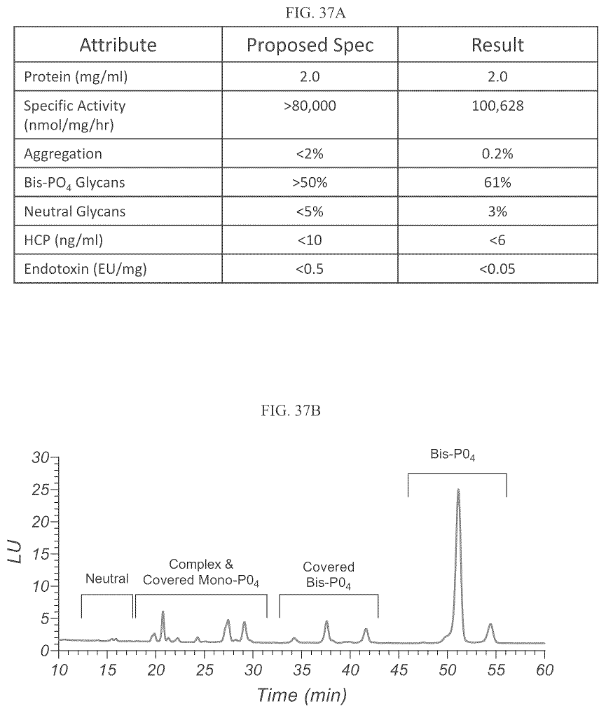

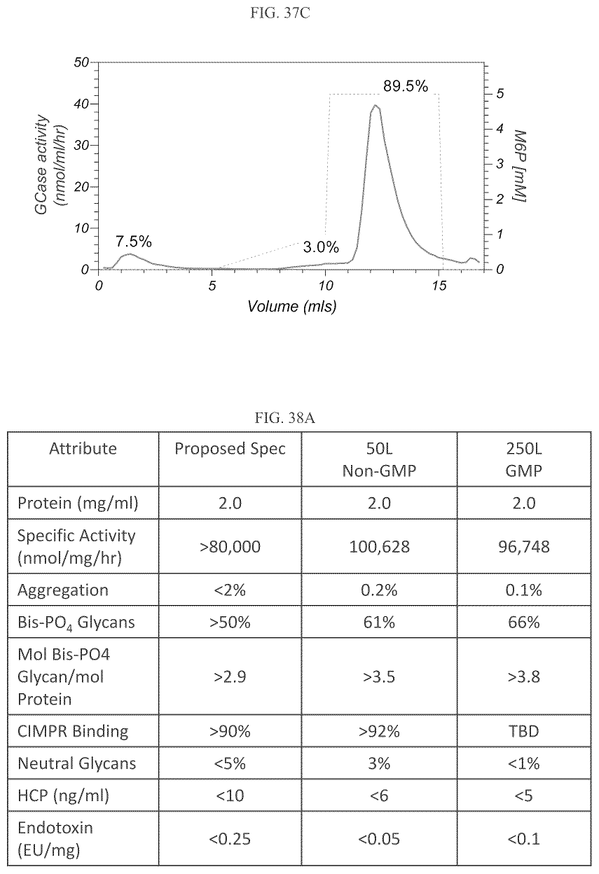

As depicted in FIG. 37A, the 50 L M011 lot resulted in specific activity of over 100,000 nmol/mg/hr, 0.2% aggregation, 61% bis phosphorylated glycans, 3% neutral glycans, less than 6 ng/ml HCP, and less than 0.05 EU/mg endotoxin. FIG. 37B depicts the glycan profiling for the 50 L M011 lot. FIG. 37C depicts the CIMPR affinity chromatography for the 50 L M011 lot.

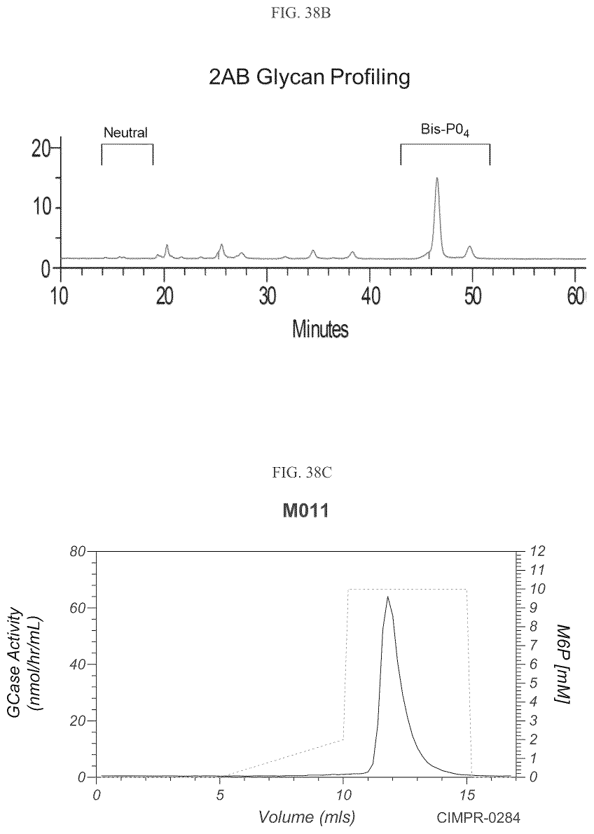

As depicted in FIG. 38A, the 250 L M011 lot resulted in specific activity of 96,748 nmol/mg/hr, 0.1% aggregation, 66% bis phosphorylated glycans, less than 1% neutral glycans, less than 5 ng/ml HCP, and less than 0.1 EU/mg endotoxin. FIG. 38B depicts the glycan profiling for the 250 L M011 lot. FIG. 38C depicts the CIMPR affinity chromatography for the 250 L M011 lot.

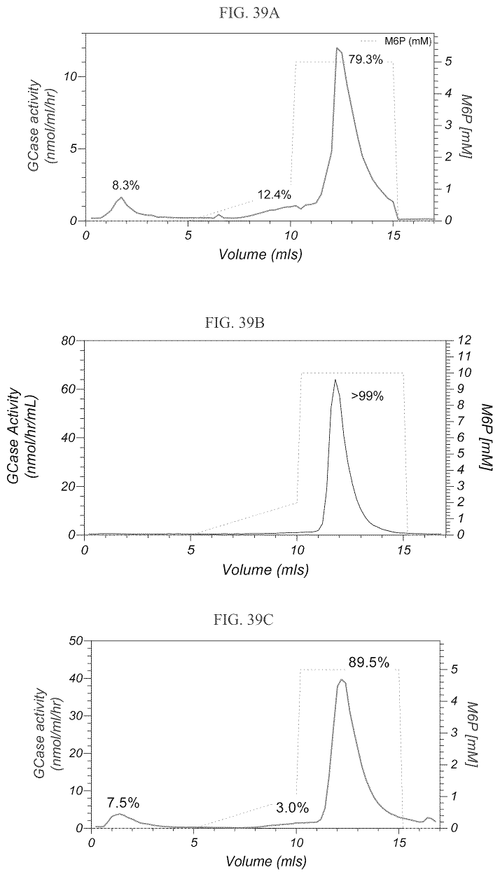

As depicted in FIGS. 39A-C, the CIMPR affinity profiles of M011 expressed in Expi293 (FIG. 39A), a stable clonal pool in CHO cells (FIG. 39B), and a final CHO clone (FIG. 39C), have been consistent in demonstrating high CIMPR affinity regardless of the cell type used.

### Example 7: Single Dose PK/PD Study in D409V/Null Gaucher Mouse Model

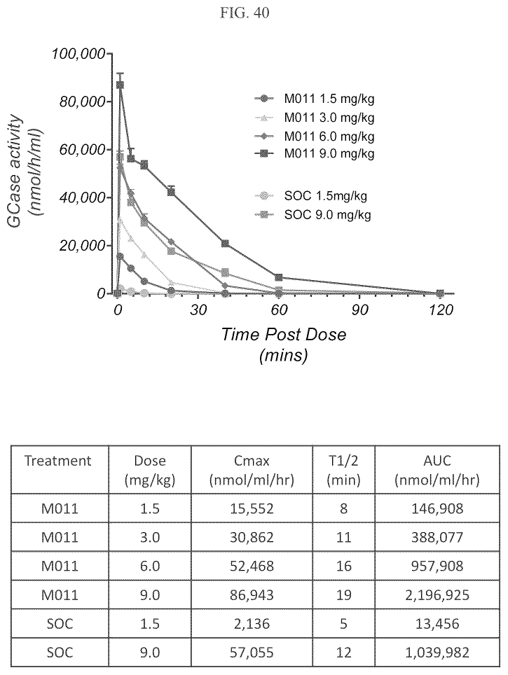

A dose range study in the D409V/null Gaucher mouse model was performed using M011 produced from a stable cell line from WuXi Biologics. Briefly, D409V/null mice were dosed once with 1.5, 3, 6, or 9 mg/kg M011, or 1.5 or 9 mg/kg imiglucerase. Two hours following dosage, analysis was conducted on GCase activity and tissue uptake. FIG. 40 depicts the GCase activity in blood serum from the mice, illustrating that M011 at 9 mg/kg dosage out-performed standard of care imiglucerase at either 1.5 or 9 mg/kg.

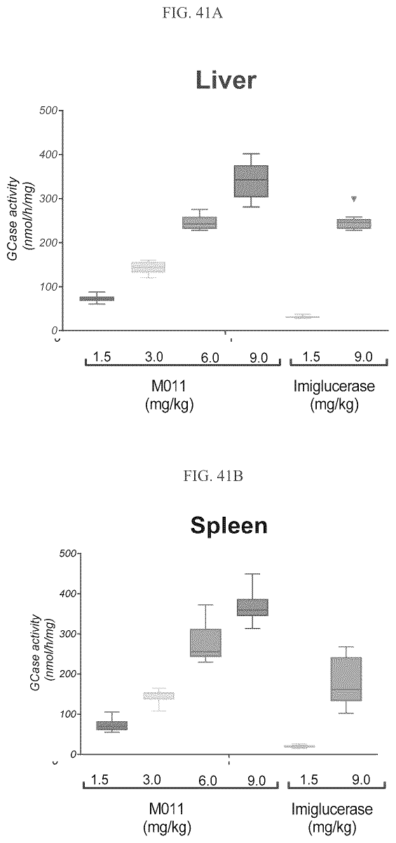

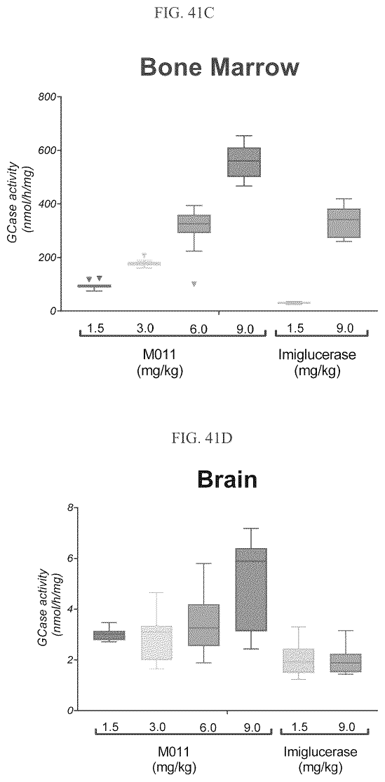

FIG. 41A-D illustrates that M011 had higher tissue uptake compared to imiglucerase in liver (FIG. 41A), spleen (FIG. 41B), bone marrow (FIG. 41C), and brain (FIG. 41D).

1. A composition comprising glucocerebrosidase, wherein at least 50% of the N-linked glycans on glucocerebrosidase are phosphorylated.

2. The composition of claim 1, wherein at least 25% of the N-linked glycans on glucocerebrosidase are phosphorylated.

3. The composition of claim 1, wherein at least 50% of the N-linked glycans on glucocerebrosidase are bis-phosphorylated.

4. The composition of claim 1, wherein less than 10% of the N-linked glycans on glucocerebrosidase are non-phosphorylated glycans.

5. The composition of claim 1, wherein less than 50% of the N-linked glycans on glucocerebrosidase are complex type N-glycans.

6. The composition of claim 1, wherein less than 5% of the N-linked glycans on glucocerebrosidase are hybrid type N-glycans.

7. The composition of any of claim 1, wherein the composition comprises less than 5 nanomolar affinity for CIMPR.

8. The composition of claim 1, wherein the composition comprises from about 1 to about 50-fold greater activity than imiglucerase.

9. The composition of claim 1, wherein the composition comprises from about 1 to about 50-fold greater activity than imiglucerase in at least one tissue selected from the group consisting of bone, bone marrow, spine, brain, spleen, liver, diaphragm, triceps, and quad.

10. The composition of claim 1, wherein the composition comprises from about 0.1 to 5-fold greater ability to reduce a substrate compared to imiglucerase.

11. The composition of claim 10, wherein the substrate is selected from the group consisting of is Lyso-GL1, lyso-GB1, C18 glucocerebroside, C24 glucocerebroside, and 1-β-D-Glucosylsphingosine.

12. The composition of claim 1, wherein the composition comprises from about 0.1 to 5-fold greater ability to reduce a substrate compared to imiglucerase in at least one tissue selected from the group consisting of serum, liver, spleen, bone marrow, bone, lung, diaphragm, quad, spine, and triceps.

13. The composition of claim 12, wherein the substrate is selected from the group consisting of is Lyso-GL1, lyso-GB1, C18 glucocerebroside, C24 glucocerebroside, and 1-β-D-Glucosylsphingosine.

14. A method of treating Gaucher disease, the method comprising administering to a subject an effective amount of the composition of claim 1.

15. The method of claim 14, wherein administering comprises enzyme replacement therapy.
